(19) United States
(12) Patent Application Publication
(10) Pub. No.: US 2020/0230595 A1
(43) Pub. Date: Jul. 23, 2020

(54) Immunodetection Method

(71) Applicants: Feng Chia University, Taichung (TW); Siward Crystal Technology, Taichung City (TW)

(72) Inventors: Chih-Hsin Shih, Taichung (TW); Ho-Chin Wu, Taichung (TW); Cheng-Liang Lee, Taichung (TW); Chuen-Yuan Hsu, Taichung City (TW)

(73) Assignees: Feng Chia University; Siward Crystal Technology

(21) Appl. No.: 16/738,580

(22) Filed: Jan. 9, 2020

(30) Foreign Application Priority Data
Jan. 17, 2019 (TW) 108101856

(51) Int. Cl. B01L 3/00; G01N 33/557; G01N 33/53; G01N 33/543

(52) U.S. Cl. CPC B01L 3/50273; B01L 3/502715; G01N 33/557; G01N 33/5304; B82Y 5/00; B01L 2300/0803; B01L 2300/0867; B01L 2400/0409; G01N 33/54346

(57) ABSTRACT

An immunodetection method is provided, including: providing a disk; providing a capture antibody on a substrate adding a sample to a reservoir; applying a first rotational speed to transfer the sample containing an antigen from the reservoir to a reaction chamber applying a second rotational speed to precipitate the sample on the substrate so as to combine the antigen in the sample with the capture antibody to obtain a first complex; using capillary force to make the sample flow out of the reaction chamber and to fill the flow channel; applying a third rotational speed to transfer the sample from the flow channel to the waste chamber; providing a detection antibody on the substrate to combine the detection antibody with the first complex to obtain a second complex; and detecting a spectral signal from the localized surface plasma resonance of the second complex.

### CROSS-REFFERENCE TO RELATED APPLICATION

This application claims priority from Taiwan Patent Application No. 108101856, filed on Jan. 17, 2019, in the Taiwan Intellectual Property Office, the content of which is hereby incorporated by reference in its entirety for all purposes.

### BACKGROUND OF THE INVENTION

### 1. Field of the Invention

The present invention relates to an immunodetection method, more particularly to an immunodetection method for a localized surface plasma resonance that may simplify the steps of injecting reagents.

### 2. Description of the Related Art

Life spans of human beings have been extended thanks to advances in medical technology. Providing a detection technique to assist in diagnosing medical data are considered essential in various medical technologies. Currently, detection techniques and instruments are provided in large hospitals. Subjects need to go to hospitals specially and undergo a lengthy testing process, resulting in a decrease in the subject's willingness to take the test.

Therefore, Point-of-Care Testing, which enables faster detection, has emerged. Specifically, a microfluidic disk is regarded as a mainstream product since the microfluidic disk can help minimize various detecting instruments, is easy to carry and operate, and has high identification. In the meantime, the microfluidic disk can be used together with a centrifugal platform to reduce reaction time.

For instance, an enzyme-linked immunosorbent assay (ELISA) can be combined with a CD ELISA of the microfluidic disk to sequentially release liquid and minimize systems, thus reducing overall detection time. Nevertheless, the CD ELISA has the disadvantages of insufficient sensitivity and complicated steps of injecting reagents. Hence, there is still a need to provide an immunodetection method that can simplify the steps of injecting reagents.

### SUMMARY OF THE INVENTION

In view of the aforementioned problem, the present invention provides an immunodetection method combined with the techniques of an immunoassay, a microfluidic disk, and a localized surface plasma resonance (LSPR) that amplifies a spectral signal. This method is also realized through an interation of microfluidic function with program automation in an effort to control the rotational speed, which achieve the simplification of the injecting process, and enables automated injection and liquid transfer. In the conventional methods which utilize centrifugal sedimentation to increase the reaction efficiency between the reagent and the substrate, a microtiter plate is placed in a large centrifuge for spinning. However, additional equipment and specialized rotor machine will be required in such methods. On the other hand, the method of the present invention only takes one motor to complete all of the testing procedures which includes centrifugal settling without additional equipment. Hence, costs may be reduced and the competitive capability of products may also be enhanced.

The purpose of the present invention is to provide an immunodetection method, including: providing a disk, the disk comprising a reservoir and a plurality of disk units, and each of the disk units including: a reaction chamber connected to a waste chamber via a flow channel, and a substrate disposed in the reaction chamber, and the substrate encapsulating a plurality of first nanoparticles; providing a capture antibody on the substrate; adding a sample to the reservoir; applying a first rotational speed to transfer the sample in the reservoir to the reaction chamber; applying a second rotational speed to precipitate the sample on the substrate so as to combine the antigen in the sample with the capture antibody to obtain a first complex; using capillary force to draw the sample from the reaction chamber and fill the flow channel; applying a third rotational speed to transfer the sample from the flow channel to the waste chamber; providing a detection antibody on the substrate to combine the detection antibody with the first complex to obtain a second complex; and detecting a spectral signal of the second complex, wherein the spectral signal is generated from a localized surface plasma resonance generated by a vibration of the plurality of first nanoparticles. Wherein, the second rotational speed is greater than the first rotational speed.

The immunodetection method of the present invention has the following advantages:

(1) The method of the present invention effectively and adjustably injects reagents by changing the balance between the centrifugal force and the capillary force. Compared to methods requiring repeated injection of multiple reagents into a microtiter plate by manpower, the present method is capable of simplifying the manufacturing process, reducing personal error and process time, while also lowering the cost. Meanwhile, in a conventional design of disks, a reaction chamber and a disk unit are disposed on the same plane; this design, however, is prone to problems such as poor air discharge capabilities. However, the reaction chamber of the present invention is protruded and disposed along the radial direction, thus effectively exhausting air through the spatial structure. The present invention also adjusts the width of the flow channel connected between the disk unit and the reaction chamber on the plane so that the disk has the advantages of smooth air exhaust and small volume required for the reagents.

(2) The method of the present invention utilizes a motor to control the rotational speed to completely remove and clean liquid in the reaction chamber. Moreover, with the method using the centrifugal force and capillary force to empty liquid, the surface of the substrate may maintain a near-dried state. This may allow accurate and stable data to be obtained when the following optical detection proceeds.

### BRIEF DESCRIPTION OF THE DRAWINGS

FIG. 1 depicts a schematic diagram of the disk according to a preferred embodiment of the present invention.

FIG. 2 depicts a partially enlarged diagram according to a preferred embodiment of the present invention.

FIG. 3 depicts a disk aspect diagram according to a preferred embodiment of the present invention.

FIG. 4 depicts a schematic diagram of the disk according to a preferred embodiment of the present invention.

FIG. 5 depicts a flow diagram of cleaning the disk according to a preferred embodiment of the present invention.

FIG. 6 depicts a flow diagram of reaction according to a preferred embodiment of the present invention.

FIG. 7 depicts a schematic diagram of the aliquot sample according to a preferred embodiment of the present invention.

FIG. 8 depicts an analytic graph of the aliquot sample of the present invention.

FIG. 9 depicts an analytic graph of the aliquot sample of the present invention.

FIG. 10 depicts an analytic graph of the angular acceleration of the aliquot sample of the present invention.

FIG. 11 depicts an analytic graph of the coefficient of variation of the aliquot sample of the present invention.

FIG. 12 depicts a schematic diagram of a quantitative apparatus of the aliquot sample of the present invention.

FIG. 13 depicts a flow channel schematic diagram of filling liquid of the present invention.

FIG. 14 depicts a rotational speed analytic graph of filling liquid of the present invention.

FIG. 15 depicts a flow image analytic graph of filling liquid of the present invention.

FIG. 16 depicts a reaction chamber image diagram of filling liquid of the present invention.

FIG. 17 depicts a flow channel schematic diagram of liquid emptying of the present invention.

FIG. 18 depicts an analytic graph of liquid emptying of the present invention.

FIG. 19 depicts a setup schematic diagram of the siphon channel of the present invention.

FIG. 20 depicts a critical rotational speed analytic graph of the siphon channel of the present invention.

FIG. 21 depicts an image diagram of residual moisture of the siphon channel of the present invention.

FIG. 22 depicts an analytic graph of residual moisture of the siphon channel of the present invention.

FIG. 23 depicts a schematic diagram of the area range detection of the present invention.

FIG. 24 depicts an analytic graph of the rotational speed of incubation of the present invention.

FIG. 25 depicts an analytic graph of the rotational speed of incubation of the present invention.

FIG. 26 depicts an analytic graph of the number of cleaning times of the present invention.

FIG. 27 depicts an analytic graph of precision of the present invention.

### DESCRIPTION OF THE PREFERRED EMBODIMENTS

To make the aforementioned purpose, the technical features, and the gains after actual implementation more obvious and understandable, the following description shall be explained in more detail with reference to the preferred embodiments together with related drawings.

In an embodiment, a disk is provided, including a reservoir and a plurality of disk units. The number of disk units may be eight or more. Each of the disk units may include a reaction chamber connected to a waste chamber via a flow channel, and a substrate disposed in the reaction chamber. Each of the disk units may include a storage chamber. A plurality of first nanoparticles may be disposed on the substrate. The plurality of first nanoparticles may be gold nanoparticles or silver nanoparticles. An extended direction of the first part of the flow channel connected to the waste chamber passes through a circle center of the disk. A capture antibody may be provided on the substrate.

In an embodiment, a first rotational speed may be applied. The centrifugal force generated by the first rotational speed is used to drive the pressure difference between the centrifugal pressure of the disk and the surface tension of the sample so that the sample is transferred from the reservoir to the reaction chamber. The sample may include an antigen. A second rotational speed may be applied so that the centrifugal force generated by the second rotational speed is used to precipitate the sample on the substrate to make the sample have contact with the capture antibody so as to specifically combine the antigen in the sample with the capture antibody to obtain the first complex. Capillary force may be used to make the sample flow out of the reaction chamber and to fill the flow channel; however, the sample does not overflow out of the flow channel, meaning that the sample has not been transferred to the waste chamber. A third rotational speed may be applied so that the angular acceleration of the third rotational speed caused by the Euler force is used to transfer the sample from the flow channel to the waste chamber.

In an embodiment, a detection antibody may be provided on the substrate to specifically combine the detection antibody with the first complex to obtain a second complex; thus, a spectral signal of the second complex from a localized surface plasma resonance may be detected. The plurality of first nanoparticles may vibrate to generate a localized surface plasma resonance. The detection antibody includes a plurality of second nanoparticles, and the plurality of first nanoparticles and the plurality of second nanoparticles may vibrate to shift a wavelength of the spectral signal and amplify the spectral signal so as to enhance the sensitivity of the spectral signal.

After the capture antibody, the sample, and the detection antibody are transferred, the cleaning solution may be transferred to remove the residual liquid, thus enhancing the precision of the detecting method.

In an embodiment, a step of providing a barrier agent on the substrate may be provided between the step of providing the capture antibody on the substrate and the step of providing the detection antibody on the substrate. The capture antibody, the antigen, the detection antibody, the cleaning solution, and the barrier agent may be the capture antibody, the antigen, the detection antibody, the cleaning solution, and the barrier agent known to a person of ordinary skill in the art.

The selected immunodetection medications are presented as follows in a preferred embodiment of the present invention:
(A) Monoclonal mouse anti-cardiac troponin I, HyTest/19C7, and concentration: 3.6 mg/ml
(B) Recombinant human cardiac troponin I, HyTest/8RTI7, and concentration: 0.9 mg/ml
(C) Monoclonal mouse anti-cardiac troponin I, HyTest/16A11, and concentration: 8.3 mg/ml
(D) Phosphate buffered saline buffer (PBS buffer)
(E) 10× Phosphate buffered saline buffer (10 X PBS), including: 80 g/L NaCl, 2 g/L KC1, 14.4 g/L NaHPO4, and 2.4 g/L KH2PO4.
(F) Surface active agent Tween-20
(G) Phosphate Buffered Saline Tween-20 (PBST), including 1× PBS and Tween-20.
(H) Edible pigment

In a preferred embodiment of the present invention, the material for the microfluidic disk is presented as follows.
(A) A biological compatible tape (3M, USA)
(B) A polycarbonate (PC) sector detection plate (KINGLAND Industrial Co., Ltd., Taiwan)
(C) A glass substrate with gold nanoparticles (SIWARD Crystal Technology Co., Ltd., Taiwan)
(D) A microtiter plate (SIWARD Crystal Technology Co., Ltd., Taiwan)

In a preferred embodiment of the present invention, the operational procedure in detail is exemplified as follows.

Preparation and synthesis

A circular glass plate with an area of 0.07 cm² is selected as a substrate, and the concentration of the capture antibody is selected to be 3000 ng/cm². In addition, the concentration of the antigen sequences are being diluted to 0, 64, 320, 1600, 8000, 40000, and 200000 pg/mL. After 1 mM and 20 mL of the tetrachloroauric acid solution is heated to boil, 38.8 mM and 2 mL of the sodium citrate solution is immediately added. Afterward, the solution is heated until the color becomes red. The gold nanoparticles (Au NPs) are then obtained. Every 100 μL of AuNPs is mixed with 1 μL of horseradish peroxidase (SA-HRP) with a concentration of 1 mg/ml to obtain horseradish peroxidase-gold nanoparticles (SA-HRP@Au NPs).

The design of the disk

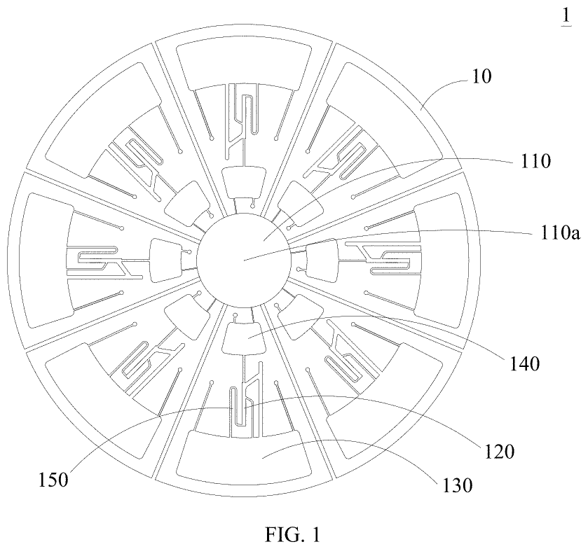

Please refer to FIG. 1. The disk 1 has a reservoir 110 and eight disk units 100 disposed in the center of the disk. When the disk 1 is circular, the disk has a circle center 110a. The reservoir 110 is connected to all of the disk units 100 for reagent aliquoting. The disk unit 100 has a reaction chamber 120, a waste chamber 130, a storage chamber 140, and a flow channel 150. A substrate coated with gold nanoparticles is provided in the reaction chamber 120. First, reagents are temporarily stored in the storage chamber 140. Each reagent is then reacted in the reaction chamber 120 by centrifugal force. The reagents flow out of the reaction chamber and fill the flow channel 150 by capillary phenomena. Afterward, the reagents are transferred to the waste chamber 130 by centrifugal force. Therefore, a user only needs to inject the reagents for once to make the reagents automatically distributed to a plurality of reaction chambers 120 in an equal amount, so that the reagents are retained in the reaction chamber 120 for incubation and coated on the substrate.

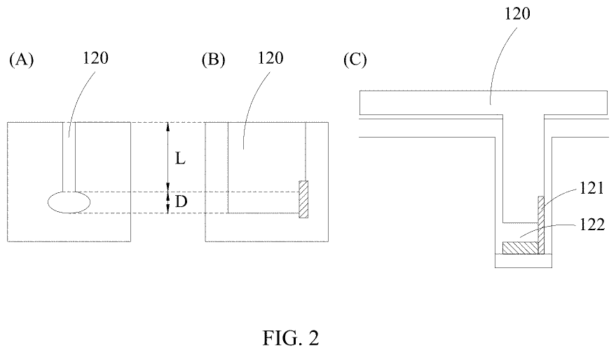

Please refer to FIG. 2. (A) is a front view of the reaction chamber, and (B) and (C) is a side view of the reaction chamber. Length L is the minimum length that may be detected by rotation on a centrifugal platform. The length D is the diameter of the detection area. The length L may be 1 mm or more. When less than 1 mm, detection by rotation may not be performed; when more than 10 mm, the negative effect of using a large amount of reagent volume then occurs. A substrate 121 is disposed in the reaction chamber 120 to form a reaction area 122.

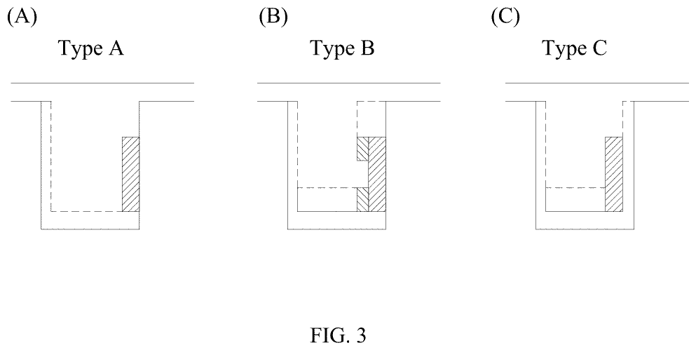

Please refer to FIG. 3. Because the substrate may be disposed at different places in the reaction chamber, the effects of the disposition places on detection are respectively compared according to three aspects such as Type A, Type B, and Type C.

TABLE 1

| | Type A | Type B | Type C |
|---|---|---|---|
| Required volume | More | Less | Less |
| for reagents | | | |
| Effects of draining | No residual | Having residual | No residual |
| reagents | liquid | liquid | liquid |
| Detection results | Having an | Not obviously having | Having an |
| | advantage | an advantage | advantage |

Please refer to Table 1, it is known that the disposition method of Type C needs less reagent volume and easily drains reagents without the residual liquid. Hence, the detection result has an advantage. Therefore, Type C is selected.

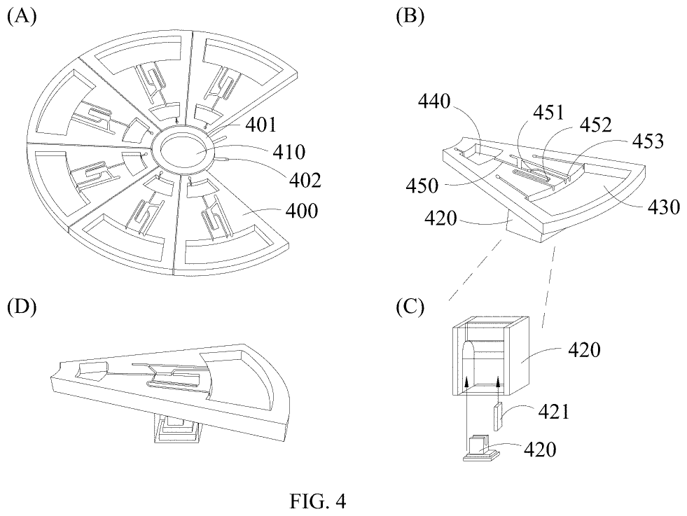

Following the description above, PMMA processed by a carving machine is provided to make PMMA on the flow channel so as to obtain a disk unit. Please refer to FIG. 4. (A) is a schematic diagram of the disk, (B) is a schematic diagram of the disk unit, (C) is a partially enlarged diagram of the disk unit, and (D) is the real-product diagram of the disc unit.

As shown in (A), the disk includes an aliquot plate 401 and eight disk units 400. The aliquot plate 401 includes a central reservoir 410. Eight capillaries 402 are disposed on the periphery of the aliquot plate 401. The capillaries 402 are connected to the disk units 400 and deliver the aliquoted liquid to the disc units 400. As shown in (B), the disk unit 400 includes a planar microfluidic structure which protrudes along the axial direction and a reaction chamber 420. The planar microfluidic structure includes a storage chamber 440, a connecting channel 452 connected to the reaction chamber, a measuring channel 451, a siphon channel 450, an air outlet channel 453, and a waste chamber 430. As shown in (C), the reaction chamber 420 disposed in the radial direction includes a glass substrate 421 coated with gold nanoparticles and a plastic cap 423.

### COMPARATIVE EXAMPLE

The glass substrate coated with gold nanoparticles may be used to replace the transparent plastic material of the microtiter plate at the bottom of the microtiter plate with the subsequent immunoadsorption reaction. The description is exemplified as follows:

(1) Microwaved with 60W for 30 minutes, the capture antibody is coated on the glass with gold nanoparticles. Then, the reagents are removed for cleaning for 4 times.

(2) Detection antibody labeled with Bovine serum albumin (BSA), an antigen, and gold nanoparticles are sequentially added into the microtiter plate. Rotated in a high rotational speed of 2700 rpm for 20 minutes, the material is deposited on the glass of the bottom of the titer plate. After the deposition, the reagents are removed and washed for 4 times.

(3) The titer plate is then turned 180 degrees and dried through high-speed centrifugation to reduce water stains on the surface. Then, an optical measurement is performed at a wavelength of 550 nm.

### EXAMPLE

The LSPR technology is combined with a microfluidic centrifugal platform. Centrifugation, precipitation, and removal of the reagent are performed on the platform. Then, detection is conducted in an optical instrument. The steps in detail is illustrated as follows:

(1) Coating Step

The capture antibody is transferred to the reaction chamber at a low rotational speed of 1000 rpm, and is coated on the glass with gold nanoparticles through microwaving at 60 W for 30 minutes. The rotational speed is increased to 2700 rpm to remove the uncombined capture antibody to the waste chamber. Then, PBST as a cleaning solution is added to the storage chamber. The reagents are driven to the reaction chamber for cleaning at a low rotational speed of 1000 rpm. After the cleaning, PBST passes through the siphon channel. Finally, the rotational speed is increased to 2700 rpm and drained the PBST to the waste chamber.

(2) Incubation and Cleaning Steps:

The bovine serum albumin is transferred to the reaction chamber by centrifugation at 1000 rpm and precipitated onto the glass by centrifugation at 2700 rpm for 20 minutes. When finished, the uncombined bovine serum albumin is drained to the waste chamber with the increase of the speed to 2700 rpm. PBST is then added to the storage chamber and driven to the reaction chamber at a low rotational speed of 1000 rpm. Finally, the rotational speed is increased to 2700 rpm so as to drain the PBST to the waste chamber. The antigen and SA-HRP@Au NPs are separately precipitated using the same rotational speed, centrifugal time, and the number of centrifugation.

(3) Detection Step:

The disk is detected on an optical instrument at a wavelength of 550 nm.

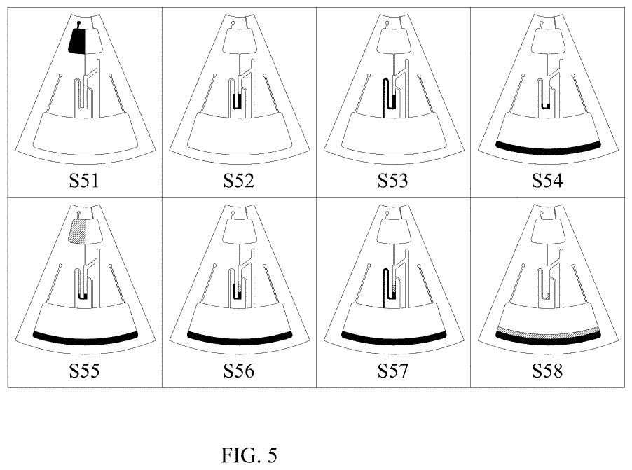

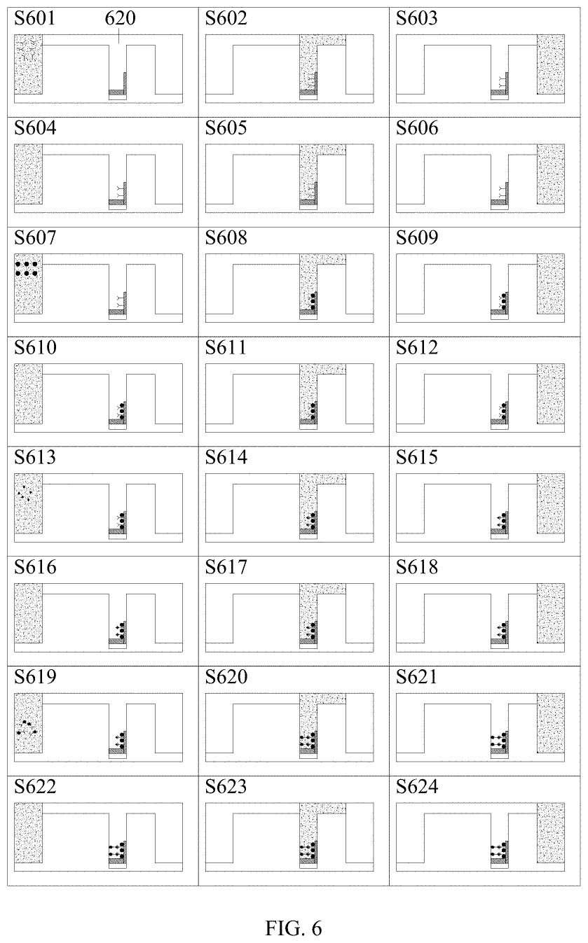

Please refer to FIG. 5 and FIG. 6. In Steps S51 to S54, the reagents are injected to fill to the full and proceed to empty the reagents. In Steps S55 to S58, the cleaning solution is injected to fill to the full and proceed to empty the cleaning solution.

In Steps S601 to S603, the capture antibody is combined onto the substrate in the reaction chamber 620. In Steps S604 to S606, the capture antibody not combined onto the substrate is removed with the use of the cleaning solution. In Steps S607 to S609, a barrier agent is provided on the substrate. In Steps S610-S612, the barrier agent not combined onto the substrate is removed with the cleaning solution. In Steps S613 to S615, the antigen is combined with the capture antibody. In steps S616 to S618, the antigen not combined with the capture antibody is removed with the cleaning solution. In Steps S619 to S621, in combination with the detection antibody and the antigen, a sandwich structure of the detection antibody, the antigen, and the capture antibody may be formed. In steps S622 to S624, the detection antibody not combined onto the antigen is removed with a cleaning solution.

Following the description above, an analysis of the comparative example and the example is performed.

Analysis One: Aliquot Sample

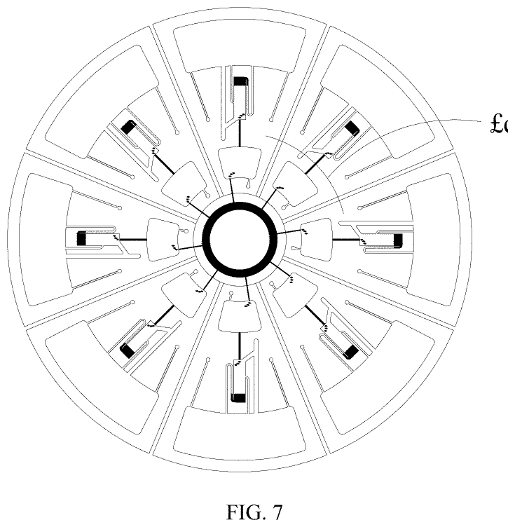

Please refer to FIG. 7, which depicts a schematic diagram of the aliquot sample according to a preferred embodiment of the present invention. One reagent needs to be injected only once. This may decrease the overall number of injection to significantly save time and manpower. When channels with different diameters are selected, the time for the liquid to break through the channels varies, and the error results from the filling in the θ direction. With the angular acceleration becoming greater, the time difference for the liquid filling the chamber and breaking through the channels becomes smaller, and the liquid volume has small variation.

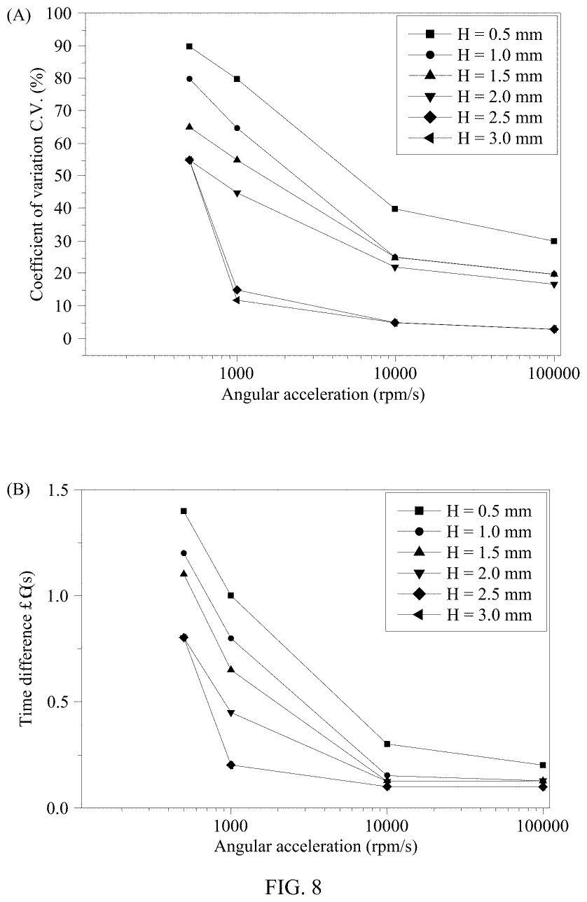

Please refer to FIG. 8. Wherein (A) is an analytic graph of the angular acceleration to the liquid of an analysis, and (B) is an analysis of the angular acceleration to the breakthrough time difference. As shown in (A), it is known that when the depth of the chamber body is 0.5 mm, the depth is small, so the resistance is large. The time for filling the central reservoir is long, and the coefficient of variation is high. As shown in (B), it is known that the time required to fill the central reservoir is short in a condition of sufficient depth of the chamber.

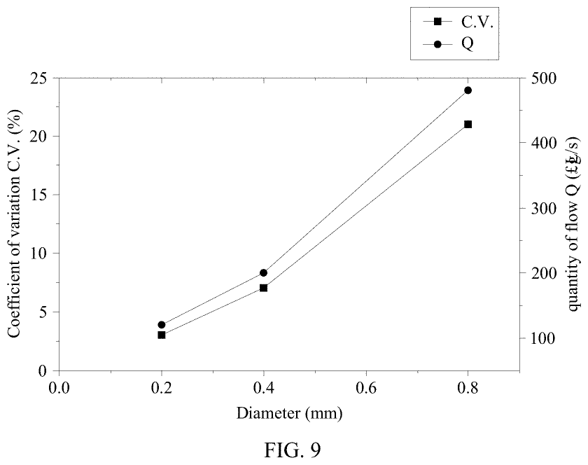

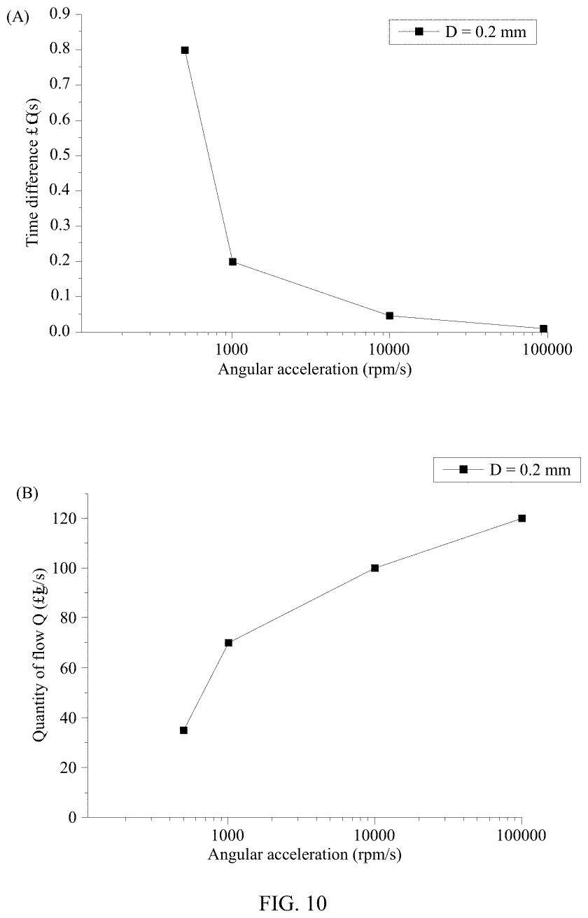

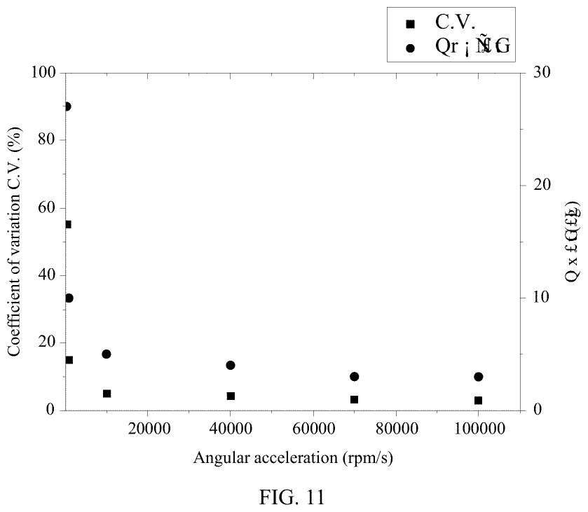

Following the description above, channels with diameters of 0.2, 0.4, and 0.8 mm are selected to perform flow error analysis. The result is presented in FIG. 9. It is known that the smaller the channel is connected, the smaller the error in the amount of volume flowing out of the first channel and the last channel may be. Therefore, the channel diameter is fixed to 0.2 mm. The breakthrough time difference, flow rate, and coefficient of variation are analyzed, and the result is shown in FIGS. 10 and 11.

Please refer to FIG. 10. (A) is an analytic graph of the breakthrough time difference of each angular acceleration. (B) is an analytic graph of the flow rate of each angular acceleration.

Please refer to FIG. 11, which depicts an analytic graph of the coefficient of variation of the aliquot sample of the present invention. The value obtained by multiplying Δt and Qr at each angular acceleration is related to the coefficient of variation C.V. The increase in the angular acceleration of the motor accelerating may reduce the time to fill the central reservoir such that the liquid may be quickly filled in the front end of the needle and flows out by breaking through. In the meantime, if a narrower channel is used, the coefficient of variation of the equal distribution of the liquid flow division may be controlled from 3 to 5%.

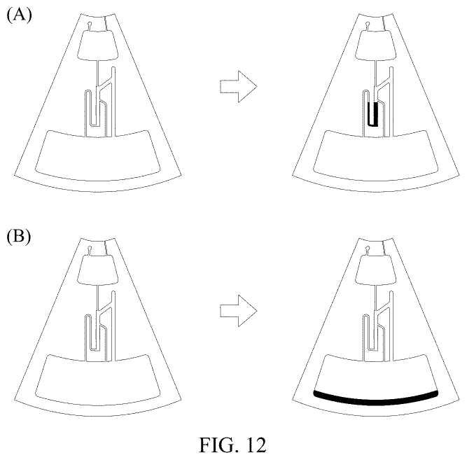

Please refer to FIG. 12. (A) and (B) are disks with and without a quantitative apparatus respectively. Due to errors existed in the aliquot, it is possible to cause all reagents to be drained to the waste chamber. Therefore, a quantitative apparatus is added next to the reaction chamber to allow excess reagents to flow to the waste chamber.

Analysis Two: Filling Liquid

If the inlet flow channel is designed to be wider and deeper, the flow channel resistance may be small with the increase of the flow rate. The liquid quickly flows into the reaction chamber. The air outlet channel is blocked, making it difficult to exhaust air. The reaction chamber is then difficult to be filled completely for accumulating the liquid. This leads to the liquid being higher in the flow channel than the siphon channel, which makes all the liquid drained into the waste chamber. Therefore, the present invention overcomes the problem of exhausting air by utilizing the reaction chamber 423 protruded in a radial direction as shown in FIG. 4 (C) and the ratio of the liquid width h to the connecting channel width H.

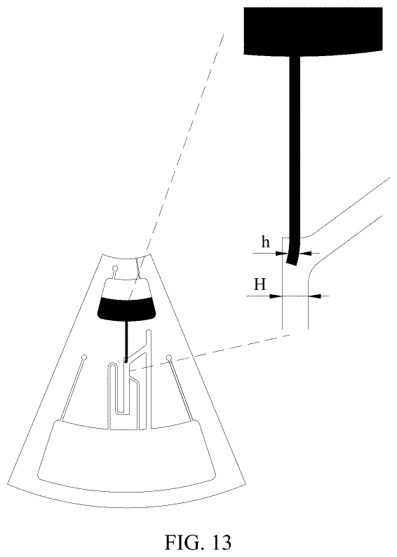

Please refer to FIG. 13. The width of the liquid flowing out of the inlet flow channel is defined as h. The width of the connecting channel is H. Different ratios (h/H) occur at different speeds. Wherein, the width of the connecting channel H refers to the inlet width of the reaction chamber.

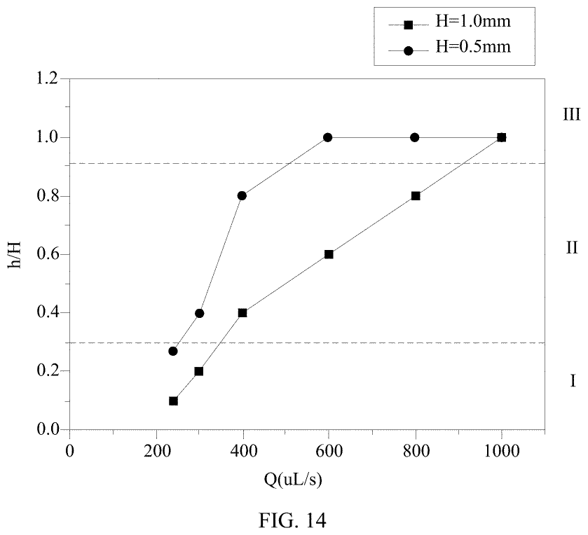

The hydraulic diameter of the inlet flow channel of the fixed liquid flowing into the reaction chamber is 0.4 mm. The rotational speeds of changes are 100, 2000, 3000, 4000, and 5000 rpm. h/H is 0.2, 0.4, 0.6, 0.8, and 1.0 mm. The flow rate at each rotational speed is investigated and the result is shown in FIG. 14.

It is known that the flow rate of volume into the reaction chamber is vitally important during the filling process. At a low flow rate of volume (Q<300), the liquid is completely filled with the reaction chamber, meaning fine air exhausting. Excess liquid passes through the measuring channel and overflows to the waste chamber. At a mid-flow rate of volume (300<Q<900), the reaction chamber is not filled with liquid due to poor air exhausting. The liquid passes through the top of the capillary and enters the waste chamber; in the meantime, excess liquid also passes through the measuring channel and then flows to the waste chamber. At a high flow rate of volume (900<Q), big bubbles preventing the liquid from flowing to the reaction chamber may be observed in the connecting channel. Most liquid flows into the waste chamber and the reaction chamber is not filled. It is known that the filling effect may change according to the fluid design as well as the ratio (h/H). If the ratio (h/H) is less than 0.3, filling may be successfully performed.

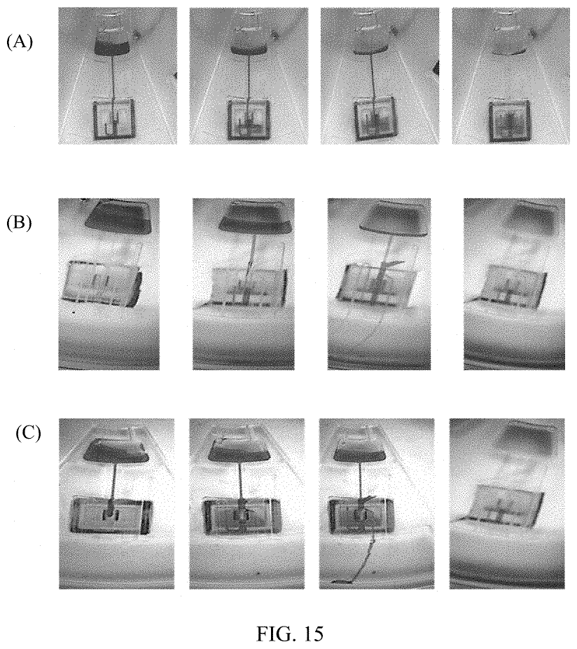

Please refer to FIG. 15. (A), (B), and (C) are flow image views respectively at the rotational speeds of 1000 rpm, 2000 rpm to 4000 rpm, and 5000 rpm.

As shown in (A), the liquid is drained out and successfully retained. As shown in (B), the bubbles occupy the volume that should be filled with liquid due to the continuous liquid, making the liquid drained to the waste chamber because of siphoning. As shown in (C), the exhaust hole valve is blocked, causing the reaction chamber not being able to be filled with the liquid. Filling the reaction chamber fails and the liquid is drained to the waste chamber.

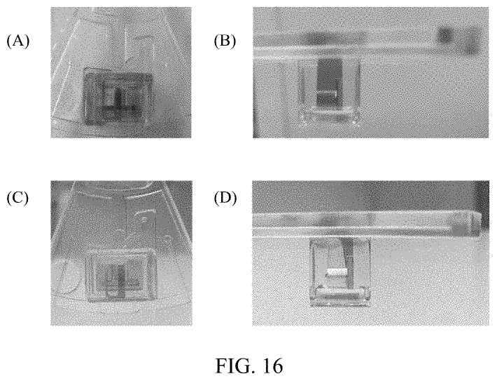

Please refer to FIG. 16. (A) and (B) respectively are top and side views of the reaction chamber at the rotational speed of 1000 rpm, and (C) and (D) respectively are top and side views of the reaction chamber at the rotational speed of 1000 rpm. When the rotational speed is 1000 rpm, the reaction chamber is filled with liquid smoothly; when the rotational speed is increased to 5000 rpm, the reaction chamber cannot be filled with liquid.

Analysis Three: Liquid Emptying

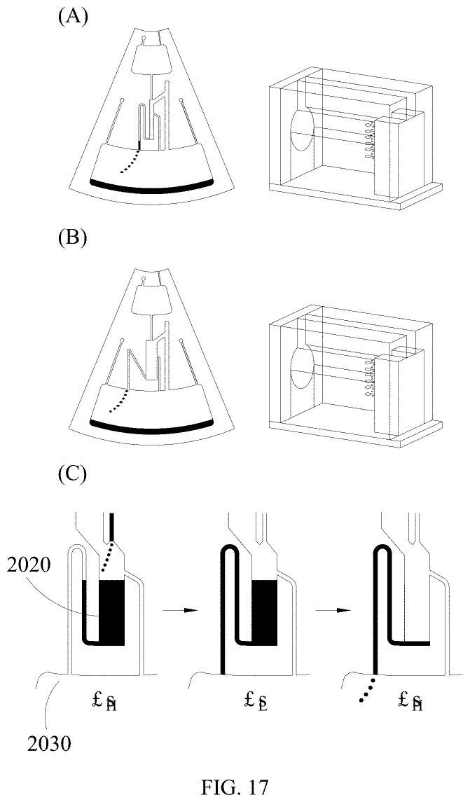

Please refer to FIG. 17. (A) is a schematic diagram of liquid emptying through a hydrophilic surface treatment. (B) is a schematic diagram of liquid emptying without a hydrophilic surface treatment. (C) is a flow channel schematic diagram of liquid emptying.

As shown in (A), the liquid automatically passes through the highest point of the siphon channel by siphoning and reaches the front of the waste chamber, and then elevates the rotational speed to make the liquid in the reaction chamber to the waste chamber, wherein the liquid in the reaction chamber is transferred with the liquid in the connecting channel. However, the effect of surface treatment fails over time. As shown in (B), to avoid the reagent entering the waste chamber directly through the siphon channel, a radial difference AR exists between the reaction chamber and the overflow point of the siphon channel. The radial difference AR is defined as the difference between the water-level balance point after centrifugation and the highest point of the siphon. A safe cumulative height is set according to the radial difference AR to prevent the liquid from directly entering the waste chamber through the siphon channel to drive the liquid. The moving distance of the liquid lifting through the surface of the unmodified channel changes with an angular acceleration of a motor and flow channel hydraulic diameter d_{H}. The liquid is lifted by momentum through changing the rotational speed. As shown in (C), the length of the flow channel has to be higher than the liquid level of the reaction chamber at a high rotational speed (ω_{H}>2000 rpm), and the width and depth are both 0.4 mm. When the motor is in high rotational speed, the liquid enters the reaction chamber due to the driving of centrifugal force. Then, the rotational speed of the motor drops to low rotational speed (ω_{L}<10 rpm), and the centrifugal force is less than the capillary force. Therefore, the flow channel will be filled with the liquid because of the driving of the capillary force. Finally, the rotational speed of the motor is elevated to high rotational speed, and the liquid in the reaction chamber is drained to the waste chamber due to the siphon effect.

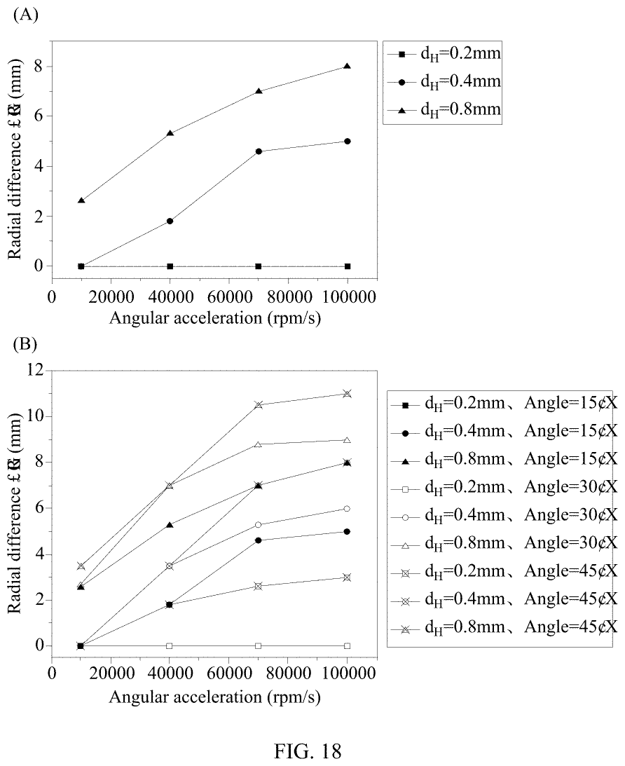

The speed is set to 4000 rpm, and then the rotational speed is reduced to 0 rpm. The angular acceleration is set to 10000, 40000, 70000, and 100000 rpm/s, and the inclination angle is set to 15, 30, 45 degrees. From the observation of the height of the liquid lifting, the result is shown in FIG. 18.

Please refer to FIG. 18. (A) is an analytic graph of angular acceleration. (B) is an analytic graph of inclination angles. It is known that higher angular acceleration produces greater Euler force, and the height of the liquid lifting also increases. The larger the hydraulic diameter is, the smaller the resistance and the greater the liquid lift will be, and vice versa. In the meantime, inclination angles affect angular acceleration required for the lift, so it is known that the larger the inclination angle is, the greater the lift of the liquid will be, and vice versa.

Even though the liquid is allowed to pass through the highest point of the siphon channel for the liquid to be drained to the waste chamber, the liquid may still be left in the flow channel connected to the front end of the waste chamber when the applied rotational speed does not reach the critical rotational speed, and causing the liquid to be directly drained to the waste chamber in the subsequent injection and liquid of centrifugation, leading to the liquid unable to be retained for the incubation of centrifugal precipitation. Therefore, the analysis of the arrangement of the siphon channel is performed.

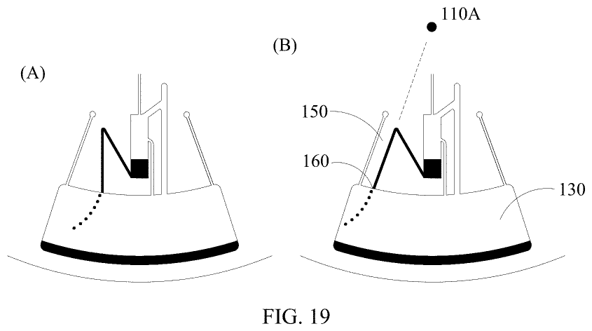

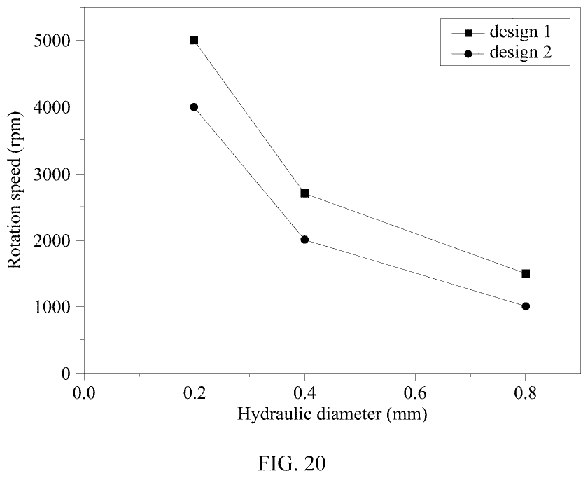

Please refer to FIG. 19. (A) and (B) respectively are the design 1 and design 2 of the siphon channel. The result of the analysis is shown in FIG. 20. When the hydraulic diameter of the siphon channel through which the liquid is emptied is larger, the critical rotational speed of the liquid emptying decreases due to a small resistance, and vice versa. In the meantime, it is known that the siphon channel of design 2 extends in the direction of centrifugation. That is, the extending direction of the end part 160 of the flow channel 150 connected to the waste chamber 130 passes through the circle center 110A of the disk. Therefore, the critical rotational speed is lower as compared to the siphon channel of design 1.

When performing LSPR sensing, the glass surface must be dry. Therefore, in the present invention, the liquid in the reaction chamber is drained to the waste chamber by the siphon channel using the aforementioned liquid-emptying method.

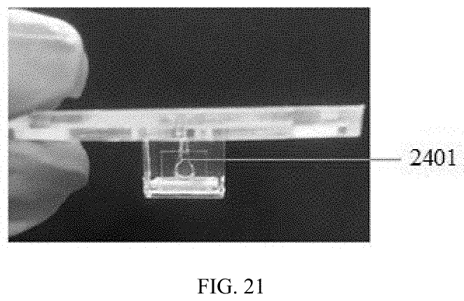

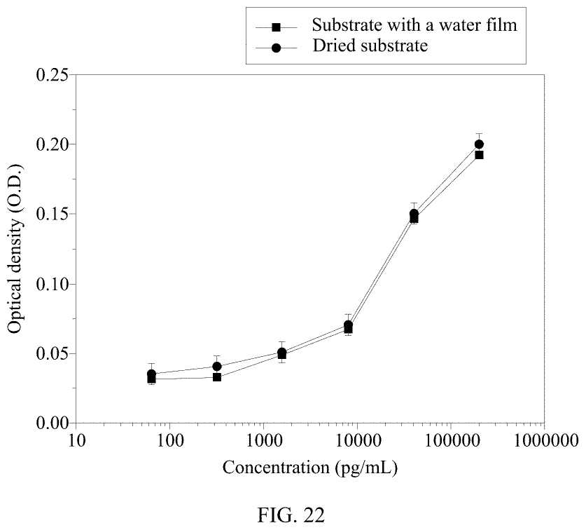

Please refer to FIG. 21. It is shown that a water film 2401 exists owing to residual moisture on the surface of the glass. Afterward, an analysis is conducted to compare a substrate with the presence of the water film and a completely dried substrate. The result is shown in FIG. 22. Although the water film only makes the signal slightly increase, the result is similar to the result with the complete drying condition.

Analysis Three: Volume of Reagents

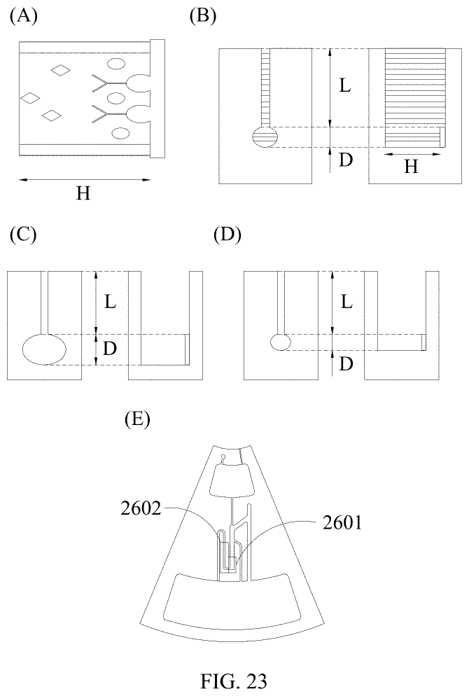

The optical density (O.D.) of Anti IgG-HRP@Au NPs is fixed. At the same concentration of STD, the more the STD volume is added, the higher the value shown in the data will be. The experiment is related to the height of the reagent vertically deposited on the bottom sensing glass. The result is shown in FIG. 23.

Please refer to FIG. 23. (A) refers to the incubation height of the reagent as the height H1. (B) refers to the specific size of an example of the present invention. Wherein, the length L of the minimum length detected by rotation on the centrifugal platform is 10 mm; the length D of the diameter of the detection area is 3 mm; the height H1 of the incubation height of the reagent is 3 mm. Wherein, the volume required for reagents Vₜₒₜₐₗ is the sum of the reaction chamber volume V_{chamber}, the connecting channel volume V_{channel}, and the flow channel volume V_{disk channel}. The reaction chamber volume V_{chamber} is a circular area multiplied by the thickness, and the length D of the diameter of the original design (as shown in (C)) is 6, whereas the length D of the new design (as shown in (D)) is 3. The connecting channel volume _{Vchannel} is a rectangular area multiplied by the thickness. The original design and the new design both have a length L of 10 and a thickness of 4. The flow channel volume V_{disk channel} is the sum of the volume of the portion 2601 and the portion 2602 (as shown in (E)). The volume of the portion 2601 is length multiplied by width and further multiplied by depth. The volume of the portion 2602 is the volume of the siphon flow channel, which is set to 1. The example of the present invention is effective in reducing the amount of reagents.

In the meantime, the reagent volume required for the comparative example and the example is analyzed; the result is shown in Table 2.

TABLE 2

| Reagent volume (μL) | Comparative example | Example |
|---|---|---|
| Capture antibody | 100 | 71 |
| Barrier agent | 100 | 71 |
| Antigen | 100 | 71 |
| Detection antibody | 100 | 71 |
| Cleaning solution | 1600 | 284 |

As shown in Table 2, it is understood that the example of the present invention may greatly reduce the volume of reagents used.

Analysis Five: Incubation Rotational Speed and Time for Reagents

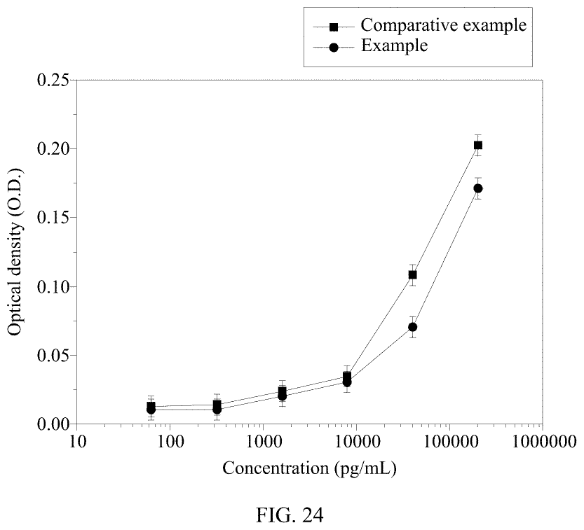

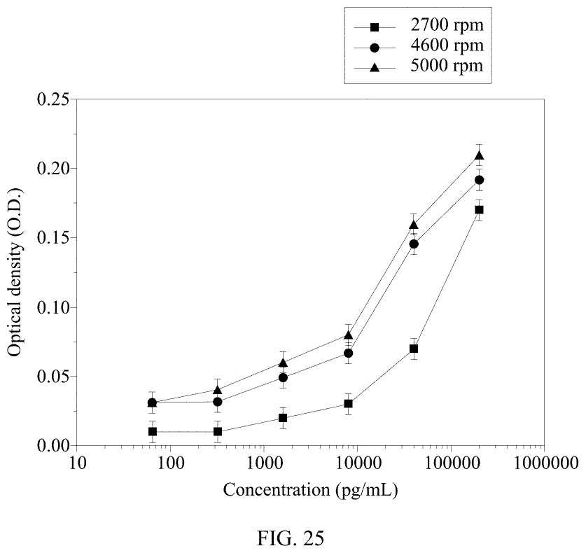

The incubation time is set to be 20 minutes. The rotational speed of 2700 rpm is set to be the optimal rotational speed for centrifugal precipitation and incubation. The antigen concentration sequence is diluted to 0, 64, 320, 1600, 8000, 40000, and 200000 pg/mL for detection on a centrifugal immunoassay analyzer. The result is shown in FIG. 24. It is understood that differences still exist in the optical density between the comparative example and example. The incubation rotational speed may be increased to improve the detection signal of the example. The result is shown in FIG. 25.

Please refer to FIG. 25. It is known that increasing the rotational speed may enhance the signal. When performing centrifugal precipitation and incubation at the rotational speed of 5000 rpm, the signal is not enhanced anymore whether the incubation time is 10 minutes or 20 minutes. Therefore, the rotational speed is set to be 5000 rpm for 10 minutes for centrifugal precipitation and incubation.

Analysis Six: Cleaning Times for Reaction

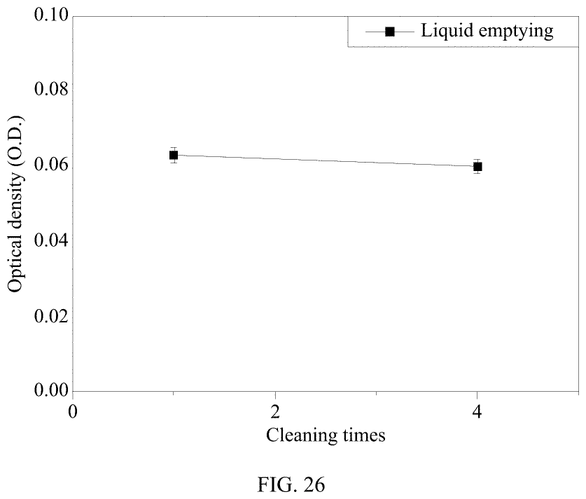

Reaction detection of the immunoadsorption method requires cleanings for several times to avoid non-specific protein adsorbed or left in the reaction chamber. Therefore, the effect of cleaning times is analyzed. The reagent is removed after precipitation and respectively cleaned for 1 time and 4 times with the cleaning solution PBST; the result is shown in FIG. 26. It is known that the signal for cleaning once is slightly larger than the signal for cleaning 4 times; however, the difference is not obvious.

Analysis Seven: Precision

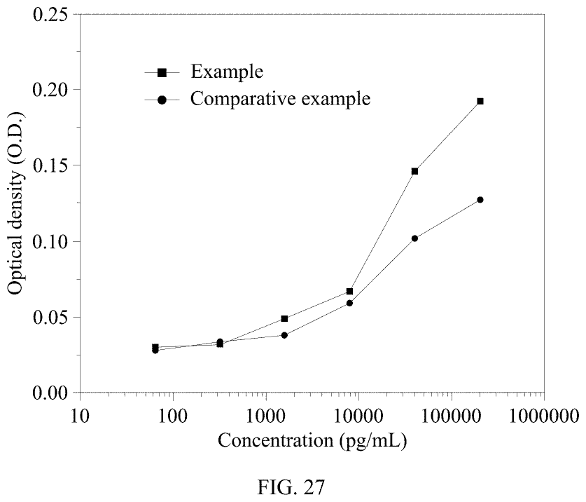

The example and comparative example of the present invention are analyzed. The result is shown in FIG. 27. It is known that the example of the present invention is similar to the comparative example, so the analytic capability of the example is similar to that of the microtiter plate.

Accordingly, the present invention has specifically described the immunodetection method by means of the embodiments and examples as mentioned above. However, it is to be understood for a person of ordinary skill in the art that modifications and changes of the embodiments may be made without departing from the technical principles of the present invention. Hence, the scope of the present invention should be as described in the following claims.

What is claimed is:

1. A immunodetection method, comprising:
providing a disk, the disk comprising a reservoir and a plurality of disk units, and each of the disk units comprising:
a reaction chamber connected to a waste chamber via a flow channel; and
a substrate disposed in the reaction chamber, and the substrate encapsulating a plurality of first nanoparticles;
providing a capture antibody on the substrate;
adding a sample comprising an antigen to the reservoir;
applying a first rotational speed to transfer the sample in the reservoir to the reaction chamber;
applying a second rotational speed to precipitate the sample on the substrate so as to combine the antigen in the sample with the capture antibody to obtain a first complex;
using capillary force to make the sample flow out of the reaction chamber and to fill the flow channel;
applying a third rotational speed to transfer the sample from the flow channel to the waste chamber;
providing a detection antibody on the substrate to combine the detection antibody with the first complex to obtain a second complex; and
detecting a spectral signal of the second complex, wherein the spectral signal being generated from a localized surface plasma resonance generated by a vibration of the plurality of first nanoparticles;
wherein, the second rotational speed is greater than the first rotational speed.

2. The immunodetection method according to claim 1, wherein the third rotational speed is greater than the first rotational speed.

3. The immunodetection method according to claim 1, wherein an extended direction of an end part of the flow channel connected to the waste chamber passes through a circle center of the disk.

4. The immunodetection method according to claim 1, wherein the detection antibody comprises a plurality of second nanoparticles, and the plurality of first nanoparticles and the plurality of second nanoparticles vibrate to shift a wavelength of the spectral signal to amplify the spectral signal.

5. The immunodetection method according to claim 1, wherein at least one of the plurality of first nanoparticles and the plurality of second nanoparticles are gold nanoparticles.

6. The immunodetection method according to claim 1, wherein the first rotational speed is less than 1500 rpm.

7. The immunodetection method according to claim 1, wherein the second rotational speed is greater than 2500 rpm.

8. The immunodetection method according to claim 1 further comprising cleaning steps, wherein the cleaning steps comprises:
applying the first rotational speed to transfer a cleaning liquid from the reservoir to the reaction chamber;
using capillary force to make the cleaning liquid flow out of the reaction chamber and to fill the flow channel; and
applying the third rotational speed to transfer the cleaning liquid from the flow channel to the waste chamber.

9. The immunodetection method according to claim 1 further comprising a step of providing a barrier agent on the substrate between the step of providing the capture antibody on the substrate and the step of providing the detection antibody on the substrate.
